(19) United States
(12) Patent Application Publication
(10) Pub. No.: US 2003/0162275 A1
(43) Pub. Date: Aug. 28, 2003

(54) Dog flavin-containing monooxygenase

(76) Inventors: Roger James Melton, Maryland Heights, MO (US); Jeffrey Charles Stevens, Kalamazoo, MI (US); Matthew J. Zaya, Paw Paw, MI (US)

Correspondence Address: PHARMACIA & UPJOHN, 301 HENRIETTA ST, 0228-32-LAW, KALAMAZOO, MI, 49007, US

(21) Appl. No.: 10/341,959

(22) Filed: Jan. 14, 2003

Related U.S. Application Data
(60) Provisional application No. 60/348,958, filed on Jan. 15, 2002.

(51) Int. Cl. C12N 9/02; C07H 21/04; C12P 21/02; C12N 15/866; C12N 5/06

(52) U.S. Cl. 435/189; 435/69.1; 435/348; 435/456; 435/320.1; 536/23.2

(57) ABSTRACT

The present invention provides an isolated, functional dog wild-type flavin-containing monooxygenase form1 (FMO1) and variants thereof; isolated polynucleotide encoding the wild-type FMO1 or a variant thereof; vector containing a polynucleotide encoding the dog wild-type FMO1 or a variant thereof; a host cell containing a said vector; a method for producing a functional dog FMO1; and a method for metabolizing a sample compound.

### CROSS REFERENCE TO OTHER APPLICATIONS

This application claims the benefit of U.S. provisional application Serial No. 60/348958, filed Jan. 15, 2002, under 35 USC 119(e)(i).

### FIELD OF THE INVENTION

The present invention relates to expression of a functional dog flavin-containing monooxygenase form 1 (FMO1) and to novel variants of the dog FMO 1.

### BACKGROUND OF THE INVENTION

Flavin-containing monooxygenases (FMOs) are an important group of xenobiotic metabolizing enzymes catalysing the oxygenation of a wide variety of xenobiotics including pharmaceuticals, agricultural chemicals, and environmental pollutants. FMOs have been identified in various organisms from bacteria to humans. Multiple FMO forms have been identified in most mammalian species. Specifically, the FMO gene family consists of five members (FMO1-5) that exhibit at least 80% amino acid identity for orthologous forms (i.e. human FMO1 and rat FMO1) and 51-58% identity for homologous forms (i.e. human FMO1 and human FMO3). The flavin-containing monooxygenase form 1 (FMO1) in human, rat, mouse, rabbit, cynomologous monkey, and pig have been published.

During the pharmaceutical discovery and development process, numerous metabolism and toxicity studies must be conducted in preclinical species (such as rat, dog and monkey) before a new chemical entity can be administered to humans. Due to their wide existence in different animals and tissues and their important metabolizing function, FMOs play an important role in studying the metabolism and toxicity of pharmaceutical agents. The beagle dog is the most common non-rodent animal used for drug preclinical safety studies. A FMO sequence of dog was disclosed online in Genbank recently; however, the procedure for conversion of the sequence to a functional protein is not taught. Lattard V., et al disclosed the cDNA sequence of dog FMO1 and the expression of said sequence using the E. coli expression vectors (pGEX-6P3); however, Lattard et al were unable to demonstrate activity for the expressed enzymes. Lattard V, et al: Cloning, sequencing, and tissue-dependent expression of flavin-containing monooxygenase (FMO) 1 and FMO3 in the dog. Drug Metabolism and Disposition, 30(2):119-127 (2002). The amino acid sequence of a dog FMO1 disclosed in GeneBank and by Lattard V et al is that of SEQ ID NO: 6 (hereinafter as dog “wild-type FMO1”). Disclosed herein is expression of a functional dog wild-type FMO1 and a novel allelic variant thereof.

### BRIEF DESCRIPTION OF SEQUENCES

SEQ ID NO: 1: Polynucleotide sequence encoding a variant of dog wild-type flavin-containing monooxygenase form 1 (FMO1), said variant having amino acid sequence of SEQ ID NO: 2.

SEQ ID NO: 2: Amino acid sequence of a variant of dog wild-type FMO1.

SEQ ID NO: 3: Polynucleotide sequence encoding a variant of dog wild-type FMO1, said variant having amino acid sequence of SEQ ID NO: 4.

SEQ ID NO: 4: Amino acid sequence of another variant of dog wild-type FMO 1.

SEQ ID NO: 5: Polynucleotide sequence encoding dog wild-type FMO1.

SEQ ID NO: 6: Amino acid sequence of dog wild-type FMO1.

### SUMMARY OF THE INVENTION

The present invention provides an isolated functional dog wild-type flavin-containing monooxygenase form 1 (FMO1); variants of dog wild-type FMO1; isolated polynucleotide encoding a variant of the dog FMO1, wherein the nucleotide molecules are RNA and DNA sequences, such as a DNA sequence coding for the amino acid sequence of SEQ ID NO: 2 or SEQ ID NO: 4; a vector containing a polynucleotide encoding dog wild-type FMO 1 or a variant thereof; a host cell containing said vector; and a method for producing a functional dog wild-type FMO1. The expressed enzyme of the present invention is useful in evaluating xenobiotic metabolism in vitro. The present invention additionally provides a cell-free extract, preferably a microsomal fraction, prepared from a transformed host cell of the invention. The present invention further provides a method for metabolizing a sample compound, which method comprises preparing a mixture of the sample compound and the host cell or cell-free extract of the invention, incubating the mixture and, optionally analyzing the products obtained thereby.

### DETAILED DESCRIPTION OF THE INVENTION

Polynucleotides of the Invention

The present invention provides isolated polynucleotides (e.g., DNA sequences and RNA transcripts, both sense and complementary antisense strands, both single and double-stranded, including splice variants thereof), which are allelic variants of dog wild-type FMO 1 gene. Examples of such polynucleotides include polynucleotides of SEQ. ID NO: 1 or SEQ ID NO: 3. Allelic variants are modified forms of a wild type gene sequence, the modification resulting from recombination during chromosomal segregation or exposure to conditions which give rise to genetic mutation. Allelic variants, like wild type genes, are naturally occurring sequences (as opposed to non-naturally occurring variants which arise from in vitro manipulation). The polynucleotides of the invention include genomic DNA, cDNA, and DNA that has been chemically synthesized in whole or in part. Genomic DNA of the invention comprises the protein coding region for a polypeptide of the invention. It is widely understood that, for many genes, genomic DNA is transcribed into RNA transcripts that undergo one or more splicing events wherein intron (i.e., non-coding regions) of the transcripts are removed, or “spliced out.” RNA transcripts that can be spliced by alternative mechanisms, and therefore be subject to removal of different RNA sequences but still encode a FMO polypeptide, are referred to in the art as splice variants which are embraced by the invention. Splice variants comprehended by the invention therefore are encoded by the same original genomic DNA sequences but arise from distinct mRNA transcripts. The invention also comprehends cDNA that is obtained through reverse transcription of an RNA polynucleotide encoding FMO (conventionally followed by second strand synthesis of a complementary strand to provide a double-stranded DNA).

Also contemplated by the invention are other polynucleotides encoding the polypeptide of SEQ ID NO: 2 or SEQ ID NO: 4, which differ in sequence from the polynucleotide of SEQ ID NO: 1 or SEQ ID NO: 3 by virtue of the well-known degeneracy of the universal genetic code. As is well known in the art, due to the degeneracy of the genetic code, there are numerous other DNA and RNA molecules that can code for the same polypeptide as that encoded by the aforementioned polypeptides. The present invention, therefore, contemplates those other DNA and RNA molecules which, on expression, encode the polypeptides of SEQ ID NO: 2 or SEQ ID NO: 4. Having identified the amino acid residue sequence encoding the dog FMO polypeptide, and with the knowledge of all triplet codons for each particular amino acid residue, it is possible to describe all such encoding RNA and DNA sequences. DNA and RNA molecules other than those specifically disclosed herein characterized simply by a change in a codon for a particular amino acid, are, therefore, within the scope of this invention.

A table of amino acids and their representative abbreviations, symbols and codons is set forth below in the following Table 1.

TABLE 1

| Amino acid | Abbrev. | Symbol | Codon(s) |
|---|---|---|---|
| Alanine | Ala | A | GCA GCC GCG GCU |
| Cysteine | Cys | C | UGC UGU |
| Aspartic acid | Asp | D | GAC GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

As is well known in the art, codons constitute triplet sequences of nucleotides in mRNA and their corresponding cDNA molecules. Codons are characterized by the base uracil (U) when present in a mRNA molecule but are characterized by base thymidine (T) when present in DNA. A simple change in a codon for the same amino acid residue within a polynucleotide will not change the sequence or structure of the encoded polypeptide. It is apparent that when a phrase stating that a particular 3 nucleotide sequence “encode(s)” any particular amino acid, the ordinarily skilled artisan would recognize that the table above provides a means of identifying the particular nucleotides at issue. By way of example, if a particular three nucleotide sequence encodes threonine the table above discloses that the possible triplet sequences are ACA, ACG, ACC and ACU (ACT if in DNA).

A polynucleotide of the present invention can be prepared by a conventional genetic engineering method, for example, cloning the subject polynucleotide from a cDNA library. The cDNA library can be obtained by preparing the mRNA fraction of suitable cells, producing cDNA therefrom using reverse transcriptase and inserting said cDNA into a vector, preferably a phage vector or a plasmid vector. Alternatively, a commercially available cDNA library derived from, for example, human liver can be employed. The library can be screened with either (i) a DNA fragment homologous to the polynucleotide or (ii) an antibody which binds to the instant polypeptide. Furthermore, the polynucleotide can be prepared by amplifying and cloning the subject polynucleotide from a cDNA library by PCR using specific oligonucleotides as primers.

The DNA fragment for screening the library can be obtained by preparing deduced oligonucleotides based on the amino acid sequence of oligopeptides from a polypeptide of the invention. Generally the deduced oligonucleotides comprise a family of possible sequences because of code degeneracy, however, it is not impossible to find oligopeptide fragments which would yield only one or two possible oligonucleotides which could encode such amino acid fragments.

The resulting oligonucleotides then can be labelled by conventional methods and used to screen the cDNA library to obtain clones carrying nucleotide sequences which encode portions or all of a polypeptide of the invention.

Alternatively, the cDNA library can be screened by an antibody which binds a polypeptide of the invention. The antibody can be polyclonal or monoclonal, made by standard techniques known in the art. Thus, a suitable host, such as a rodent is immunized with the instant enzyme, or portions thereof, using suitable diluents, routes and schedules as known in the art or which are optimized to obtain a suitable immune response thereto. At that point either serum, for polyclonal antisera, or splenocytes, for monoclonal antisera, are obtained.

Vectors, Host Cells, and Expression of the Invention

The invention also relates to vectors that comprise a polynucleotide encoding a functional dog wild-type FMO1 (polypeptide of the invention), or vectors that comprise a polynucleotide encoding a variants of dog wild-type FMO1 of the invention, host cells that are genetically engineered with vectors of the invention and the production of polypeptides of the invention by recombinant techniques. Cell-free translation systems can also be employed to produce such proteins using RNAs derived from the DNA constructs of the invention.

Autonomously replicating recombinant expression constructs such as plasmid and viral DNA vectors incorporating a polynucleotide are also provided. Expression constructs wherein a polynucleotide is operatively linked to an endogenous or exogenous expression control DNA sequence and a transcription terminator are also provided. Expression control DNA sequences include promoters, enhancers, and operators, and are generally selected based on the expression systems in which the expression construct is to be utilized. Promoter and enhancer sequences are generally selected for the ability to increase gene expression, while operator sequences are generally selected for the ability to regulate gene expression. Expression constructs of the invention may also include sequences encoding one or more selectable markers that permit identification of host cells bearing the construct. Expression constructs may also include sequences that facilitate, and preferably promote, homologous recombination in a host cell. Constructs of the invention also include sequences necessary for replication in a host cell.

For recombinant production, host cells can be genetically engineered to incorporate expression systems or portions thereof or polynucleotides of the invention. Introduction of a polynucleotide into the host cell can be effected by methods described in many standard laboratory manuals, such as Davis et al., BASIC METHODS IN MOLECULAR BIOLOGY, (1986) and Sambrook et al., MOLECULAR CLONING: A LABORATORY MANUAL, 2nd Ed., Cold Spring Harbor Laboratory Press, Cold Spring Harbor, N.Y. (1989), such as, calcium phosphate transfection, DEAE-dextran mediated transfection, transvection, microinjection, cationic lipid-mediated transfection, electroporation, transduction, scrape loading, ballistic introduction and infection.

According to one aspect of the invention, host cells are provided, including prokaryotic and eukaryotic cells, comprising a polynucleotide of the invention or a polynucleotide encoding a functional FMO1 in a manner which permits expression of the encoded polypeptide. Polynucleotides may be introduced into the host cell as part of a circular plasmid, or as linear DNA comprising an isolated protein coding region or a viral vector. Methods for introducing DNA into the host cell well known and routinely practiced in the art include transformation, transfection, electroporation, nuclear injection, or fusion with carriers such as liposomes, micelles, ghost cells, and protoplasts. Expression systems of the invention include bacterial, yeast, fungal, plant, insect, invertebrate, and mammalian cells systems.

Suitable host cells for the invention include prokaryotes, yeast, and higher eukaryotic cells. Expression vectors for use in prokaryotic hosts generally comprise one or more phenotypic selectable marker genes. Such genes generally encode, e.g., a protein that confers antibiotic resistance or that supplies an auxotrophic requirement. A wide variety of such vectors are readily available from commercial sources. Examples include pSPORT vectors, pGEM vectors (Promega), pPROEX vectors (LTI, Bethesda, Md.), Bluescript vectors (Stratagene), and pQE vectors (Qiagen).

A preferred host cell is insect cell for the invention. An example of suitable insect cell includes Sf8, Sf9, or Sf21 cells, silkworm, or a cell derived from Trichoplusia ni. In a preferred embodiment, the polypeptides of the invention are expressed using a baculovirus expression system in Sf-9 cells. Further information regarding the use of baculovirus systems for the expression of heterologous proteins in insect cells are reviewed by Luckow and Summers, Bio/Technology 6:47 (1988).

The polypeptides of the invention may also be expressed in yeast host cells from genera including Saccharomyces, Pichia, and Kluveromyces. Yeast hosts include S. cerevisiae and P. pastoris. Yeast vectors will often contain an origin of replication sequence from a 2 micron yeast plasmid, an autonomously replicating sequence (ARS), a promoter region, sequences for polyadenylation, sequences for transcription termination, and a selectable marker gene. Vectors replicable in both yeast and E. coli (termed shuttle vectors) may also be used. In addition to the above-mentioned features of yeast vectors, a shuttle vector will also include sequences for replication and selection in E. coli. Direct secretion of polypeptides of the invention expressed in yeast hosts may be accomplished by the inclusion of nucleotide sequence encoding the yeast factor leader sequence at the 5′ end of the nucleotide sequence encoding a polypeptide of the invention.

The polypeptide of the invention may also be expressed in mammalian host cells. Non-limiting examples of suitable mammalian cell lines include the COS-7 line of monkey kidney cells (Gluzman et al., Cell 23:175 (1981)), Chinese hamster ovary (CHO) cells, and human 293 cells.

The choice of a suitable expression vector for expression of the polypeptides of the invention will of course depend upon the specific host cell to be used, and is within the skill of the ordinary artisan. Examples of suitable expression vectors include pcDNA3 (Invitrogen) and pSVL (Pharmacia Biotech). Expression vectors for use in mammalian host cells may include transcriptional and translational control sequences derived from viral genomes. Commonly used promoter sequences and modifier sequences which may be used in the present invention include, but are not limited to, those derived from human cytomegalovirus (CMV), Adenovirus 2, Polyoma virus, and Simian virus 40 (SV40). Methods for the construction of mammalian expression vectors are disclosed, for example, in Okayama and Berg (Mol. Cell. Biol. 3:280 (1983)); Cosman et al. (Mol. Immunol. 23:935 (1986)); Cosman et al. (Nature 312:768 (1984)); EP-A-0367566; and WO 91/18982.

Polypeptides of the Invention

The present invention provides isolated dog functional flavin-containing monooxygenase form 1 polypeptides that consist of, consist essentially of, or comprise of the amino acid sequence of SEQ ID NO: 6, wherein the isolated FMO1is expressed using a baculovirus expression system in insect cells, which functional FMO1 polypeptides are collectively referred herein as polypeptides of the invention. All obvious variants of these polypeptides that are within the art to make and use are also contemplated in the invention. Some of these variants are described in detail below.

Accordingly, the present invention provides an isolated dog functional FMO1 polypeptide that consists of the amino acid sequence of SEQ ID NO: 6, wherein the isolated polypeptide is expressed using a baculovirus expression system in insect cells. A polypeptide consists of an amino acid sequence when the amino acid sequence is the final amino acid sequence of the polypeptide.

The present invention further provides an isolated dog functional FMO1 polypeptide that consists essentially of the amino acid sequence of SEQ ID NO: 6, wherein the isolated polypeptide is expressed using a baculovirus expression system in insect cells. A polypeptide consists essentially of an amino acid sequence when such an amino acid sequence is present with only a few additional amino acid residues, for example from about 1 to about 100 or so additional residues, typically from 1 to about 20 additional residues in the final polypeptide.

The present invention further provides an isolated dog functional FMO1 polypeptide that comprises the amino acid sequence of SEQ ID NO: 6, wherein the isolated polypeptide is expressed using a baculovirus expression system in insect cells. A polypeptide comprises an amino acid sequence when the amino acid sequence is at least part of the final amino acid sequence of the protein. In such a fashion, the polypeptide can be only the polypeptide or have additional amino acid molecules, such as amino acid residues (contiguous encoded sequence) that are naturally associated with it or heterologous amino acid residues/peptide sequences. Such a polypeptide can have a few additional amino acid residues or can comprise several hundred or more additional amino acids.

The dog flavin-containing monooxygenase form 1 having the sequence of SEQ ID NO: 6 is also referred herein to as dog wild-type FMO 1.

The polypeptides of the present invention can be attached to heterologous sequences to form chimeric or fusion proteins. Such chimeric and fusion proteins comprise a polypeptide of the invention operatively linked to a heterologous protein having an amino acid sequence not substantially homologous to the polypeptides of the invention. “Operatively linked” indicates that the polypeptides of the invention and the heterologous protein are fused in-frame. The heterologous protein can be fused to the N-terminus or C-terminus of the polypeptides of the invention. A chimeric or fusion protein can be produced by standard recombinant DNA techniques. For example, DNA fragments coding for the different protein sequences are ligated together in-frame in accordance with conventional techniques. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and re-amplified to generate a chimeric gene sequence (see Ausubel et al., Current Protocols in Molecular Biology, 1992). Moreover, many expression vectors are commercially available that already encode a fusion moiety (e.g., a GST protein).

The present invention also provides and enables obvious variants of the amino acid sequence of the polypeptides of the present invention, such as naturally occurring mature forms of the peptide, allelic/sequence variants of the peptides, non-naturally occurring recombinantly derived variants of the peptides, and orthologs and paralogs of the peptides. Such variants can readily be generated using art-known techniques in the fields of recombinant nucleic acid technology and protein biochemistry. Such variants can readily be identified/made using molecular techniques and the sequence information disclosed herein. Further, such variants can readily be distinguished from other peptides based on sequence and/or structural homology to the polypeptides of the present invention. The degree of homology/identity present will be based primarily on whether the peptide is a functional variant or non-functional variant, the amount of divergence present in the paralog family and the evolutionary distance between the orthologs.

Allelic variants of the polypeptides of the invention can readily be identified as being a dog polypeptide having a high degree (significant) of sequence homology/identity to at least a portion of the polypeptide of the invention as well as being encoded by the same genetic locus as the polypeptide provided herein. As used herein, two proteins (or a region of the proteins) have significant homology when the amino acid sequences are typically at least about 70-80%, 80-90%, and more typically at least about 90-95% or more homologous. Percent amino acid sequence “identity” with respect to the polypeptides of SEQ ID NO: 6 is defined herein as the percentage of amino acid residues in the candidate sequence that are identical with the residues in the sequence of SEQ ID NO: 6 after aligning both sequences and introducing gaps, if necessary, to achieve the maximum percent sequence identity, and not considering any conservative substitutions as part of the sequence identity. Percent sequence “homology” with respect to the polypeptide of SEQ ID NO: 6 is defined herein as the percentage of amino acid residues in the candidate sequence that are identical with the residues in the sequence SEQ ID NO: 6 after aligning the sequences and introducing gaps, if necessary, to achieve the maximum percent sequence identity, and also considering any conservative substitutions as part of the sequence identity.

Paralogs of a polypeptide of the invention can readily be identified as having some degree of significant sequence homology/identity to at least a portion of the polypeptide of the invention, as being encoded by a FMO1 gene from dogs, and as having similar activity or function. Two proteins will typically be considered paralogs when the amino acid sequences are typically at least about 60% or greater, and more typically at least about 70% or greater homology through a given region or domain.

Non-naturally occurring variants of the polypeptides of the present invention can readily be generated using recombinant techniques. Such variants include, but are not limited to deletions, additions (insertions) and substitutions in the amino acid sequence of the polypeptide of the invention. Insertions may be located at either or both termini of the protein, or may be positioned within internal regions of the polypeptide of the invention. Insertion variants with additional residues at either or both termini can include for example, fusion proteins and proteins including amino acid tags or labels. Specifically, insertion variants include polypeptide of the invention wherein one or more amino acid residues are added to the amino acid sequence, or to a biologically active fragment thereof. Insertion variants therefore can also include fusion proteins wherein the amino and/or carboxy termini of the polypeptide of the invention is fused to another polypeptide. Various tag polypeptides and their respective antibodies are well known in the art. Examples include poly-histidine (poly-his) or poly-histidine-glycine (poly-his-gly) tags; the influenza HA tag polypeptide and its antibody 12CA5 [Field et al., Mol. Cell. Biol., 8:2159-2165 (1988)]; the c-myc tag and the 8F9, 3C7, 6E10, G4, B7 and 9E10 antibodies thereto [Evan et al., Molecular and Cellular Biology, 5:3610-3616 (1985)]; and the Herpes Simplex virus glycoprotein D (gD) tag and its antibody [Paborsky et al., Protein Engineering, 3(6):547-553 (1990)]. Other tag polypeptides include the Flag-peptide [Hopp et al., BioTechnology, 6:1204-1210 (1988)]; the KT3 epitope peptide [Martin et al., Science, 255:192-194 (1992)]; an alpha -tubulin epitope peptide [Skinner et al., J. Biol. Chem., 266:15163-15166 (1991)]; and the T7 gene 10 protein peptide tag [Lutz-Freyermuth et al., Proc. Natl. Acad. Sci. USA, 87:6393-6397(1990)]. In addition, the FMO polypeptide can be tagged with enzymatic proteins such as peroxidase and alkaline phosphatase.

The invention also provides deletion variants wherein one or more amino acid residues in a polypeptide of the invention are removed. Deletions can be effected at one or both termini of the polypeptide of the invention, or with removal of one or more residues within the amino acid sequence a polypeptide of the invention. Deletion variants, therefore, include all fragments of a polypeptide of the invention.

The invention also provides deletion variants, that is, variants resulting from substitutions. For example, one class of substitutions is conserved amino acid substitution. Such substitutions are those that substitute a given amino acid in a polypeptide of the invention by another amino acid of like characteristics. Thus, as used herein the term “conservative substitution” refers to a substitution of one amino acid for another amino acid with generally similar properties such that the overall functioning of the enzyme is not seriously affected. Typically seen as conservative substitutions are the replacements, one for another, among the aliphatic amino acids Ala, Val, Leu, and Ile; interchange of the hydroxyl residues Ser and Thr; exchange of the acidic residues Asp and Glu; substitution between the amide residues Asn and Gln; exchange of the basic residues Lys and Arg; and replacements among the aromatic residues Phe and Tyr. Guidance concerning which amino acid changes are likely to be phenotypically silent are found in Bowie et al., Science 247:1306-1310 (1990). Other exemplary conservative substitutions are set out in Table 2 immediately below.

TABLE 2
Conservative Substitutions

| Original Residue | Exemplary Substitution |
|---|---|
| Ala (A) | Val, Leu, Ile |
| Arg (R) | Lys, Gln, Asn |
| Asn (N) | Gln, His, Lys, Arg |
| Asp (D) | GIu |
| Cys (C) | Ser |
| Gln (Q) | Asn |
| Glu (E) | Asp |
| His (H) | Asn, Gln, Lys, Arg |
| Ile (D) | Leu, Val, Met, Ala, Phe, |
| Leu (L) | Ile, Val, Met, Ala, Phe |
| Lys (K) | Arg, Gln, Asn |
| Met (M) | Leu, Phe, Ile |
| Phe (F) | Leu, Val, Ile, Ala |
| Pro (P) | Gly |
| Ser (S) | Thr |
| Thr (T) | Ser |
| Trp (W) | Tyr |
| Tyr (Y) | Trp, Phe, Thr, Ser |
| Val (V) | Ile, Leu, Met, Phe, Ala |

Variants of polypeptides of the invention can be fully functional or can lack function in one or more activities, e.g. ability to bind substrate. Fully functional variants typically contain only conservative variation or variation in non-critical residues or in non-critical regions. Functional variants can also contain substitution of similar amino acids that result in no change or an insignificant change in function. Alternatively, such substitutions may positively or negatively affect function to some degree.

Non-functional variants typically contain one or more non-conservative amino acid substitutions, deletions, insertions, inversions, or truncation or a substitution, insertion, inversion, or deletion in a critical residue or critical region.

Examples of particular variants of polypeptides of the invention include polypeptide of SEQ ID NO: 2 and SEQ ID NO: 4. The two variants were shown to be inactive by the enzyme assay under the testing conditions described in detail in Example 3 below. This enzyme assay represents a single known method for the detemination of enzyme activity of various FMOs. Briefly, the methyl-p-tolyl (MPT) S-oxidase activity of the expressed variants of the polypeptide was assayed as a measure of FMO function. MPT S-oxidation has been shown to be a high velocity reaction catalyzed by various FMO forms (Sadeque et al., 1992 and Rettie et al., 1994). Incubations included 25 ug of Sf-9 cell (containing the polypeptide) microsomal protein 1 mM NADPH, and 0.1 M tricine. Incubations were started by the addition of MPTS and were conducted at 37° C. for at least 10 minutes. Following termination of the incubation, the supernatant was analyzed by high performance liquid chromatography and the MPT sulfoxide product was detected by absorbance at 240 nm and quantitated using an authentic standard of the sulfoxide metabolite.

Variants of the polypeptide of the invention are useful in defining important structural features of the enzyme, in the development of models for predicting substrate interactions with FMO forms, as a means for identifying differences in pharmacokinetic, toxicokinetic, or pharmacological responses in dogs to various test drugs, which has wide application in the pharmaceutical industry, or as antigens.

The expressed polypeptides of the invention generally are present in the microsomal membrane in Eucaryotic cells where monooxygenase activity occurs, but predominately in cytosolic forms in bacteria and unicellular organisms. The expressed polypeptides can, for example, be used to investigate metabolism of a xenobiotic in vitro, preferably in the form of intact insect cells or cell-free extracts. The xenobiotic can be a pharmaceutical compound, toxic substance, carcinogen or mutagen, or can be a compound which can be converted to a toxic substance, carcinogen or mutagen, for example, in vivo. Thus the instant enzyme can be used to test the xenobiotic or product thereof for any adverse characteristics.

As the cell-free extract, for example, a microsomal fraction of transformed cells can be used. Preparation of the cell-free extract or the microsomal fraction may be performed according to a conventional method described, for example, in DNA, 4(3): 203-210 (1985).

The insect cells or the cell-free extract thus obtained may be used to analyze a metabolic pathway of a sample compound by reacting the sample compound with the insect cells or the cell-free extract. The reaction can be performed by adding the sample compound to a culture of the insect cells or to a solution of cell-free extract, such as culture medium or buffer containing the yeast cells or the cell-free extract, and by incubating the reaction mixture, for example, at a temperature of about 25 to about 37 degree C., for about 0.1 to about 48 hours. Incubations with cell-free extracts typically require the presence of the co-factor NADPH for activity to be observed and for the enzyme to avoid degradation. The amount of the insect cells, or said cell-free extract, and the amount of the sample compound to be added to the reaction mixture may be varied according to various conditions such as the reaction temperature, reaction time and the type of the sample compound. For example, the amount of the insect cells is preferably between about 10⁷ and about 10 ⁸ cells, or about 5 to about 200 ul of the microsomal fraction (per 1 ml of the solution), and the amount of the sample compound to be added to the reaction mixture is preferably between about 0.01 and about 1 micro mole per 1 ml of the solution. The amounts optionally can be increased or decreased as desired.

After completion of the reaction, analysis of the products and metabolites in the reaction solution can be conducted according to a conventional analytical method, as described in Guideline of Instrumental Analysis (New edition, first published 1985, KAGAKU-DOJIN Publishing Company, edited by Jiro Shiokawa et al.) or Spectrometric Identification of Organic Compounds (Fourth edition, third published 1984, TOKYO KAGAKU DOJIN Co., Ltd., edited by R. M. Silver et al.). As used herein, “metabolites” is meant to encompass any product resulting from an action of the instant enzyme.

Alternatively, the resulting products can be tested, for example, for toxicity or transforming activity in standard assays and bioassays, using for example, cell lines and organisms as test systems for such assessments. As used herein, “toxicity”, is meant to encompass compounds which have a harmful effect on a cell, organism and the like. Thus, for example, a carcinogen or a mutagen is toxic.

On the basis of the data obtained, it can be judged as to whether the sample compound either is detoxified or activated to a harmful compound by the present enzyme.

### Definitions and Conventions

The definitions and explanations below are for the terms as used throughout this entire document including both the specification and the claims.

As used herein a “functional dog FMO1 polypeptide” refers to an isolated polypeptide that has an activity of a dog FMO known in the art. Activities of a dog FMO includes the oxidation of nitrogen-, sulfur-, phosphorus-, or other heteroatom-containing chemicals, drugs, or pesticides. A particular example of the oxidation activity of FMO is the oxidation of methyl-p-tolyl (MPT) S-oxidation activity. An enzyme assay for determining the MPT S-oxidation activity of an expressed FMO1 polypeptide is provided in Example 3.

As used herein “polynucleotide” generally refers to any polyribonucleotide or polydeoxribonucleotide, which may be unmodified RNA or DNA or modified RNA or DNA. “Polynucleotides” include, without limitation, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA. The term “polynucleotide” also includes DNAs or RNAs containing one or more modified bases and DNAs or RNAs with backbones modified for stability or for other reasons. “Modified” bases include, for example, tritylated bases and unusual bases such as inosine. A variety of modifications may be made to DNA and RNA; thus, “polynucleotide” embraces chemically, enzymatically or metabolically modified forms of polynucleotides as typically found in nature, as well as the chemical forms of DNA and RNA characteristic of viruses and cells. “polynucleotide” also embraces relatively short polynucleotides, often referred to as oligonucleotides.

As used herein “polypeptide” refers to any peptide or protein comprising amino acids joined to each other by peptide bonds or modified peptide bonds. “polypeptide” refers to both short chains, commonly referred to as peptides, oligopeptides or oligomers, and to longer chains, generally referred to as proteins. Polypeptides may contain amino acids other than the 20 gene-encoded amino acids. “Polypeptides” include amino acid sequences modified either by natural processes, such as post-translational processing, or by chemical modification techniques which are well known in the art. Such modifications are well described in basic texts and in more detailed monographs, as well as in a voluminous research literature. Modifications may occur anywhere in a polypeptide, including the peptide backbone, the amino acid side-chains and the amino or carboxyl termini. It will be appreciated that the same type of modification may be present to the same or varying degrees at several sites in a given polypeptide. Also, a given polypeptide may contain many types of modifications. Polypeptides may be branched as a result of ubiquitination, and they may be cyclic, with or without branching. Cyclic, branched and branched cyclic polypeptides may result from post-translation natural processes or may be made by synthetic methods. Modifications or modified forms include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, proteolytic processing, phosphorylation prenylation, racemization, selenoylation, sulfation, transfer-RNA mediated addition of amino acids to proteins such as arginylation, and ubiquitination (see, for instance, Proteins-Structure and Molecular Properties, 2nd Ed., T. E. Creighton, W. H. Freeman and Company, New York, 1993; Wold, F., Post-translational Protein Modifications: Perspectives and Prospects, pgs. 1-12 in Postranslational Covalent Modification of Proteins, B. C. Johnson, Ed., Academic Press, New York, 1983; Seifter et al., “Analysis for protein modifications and nonprotein cofactors”, Meth Enzymol (1990) 182:626-646 and Rattan et al., “Protein Synthesis: Post-translational Modifications and Aging”, Ann NY Acad Sci (1992) 663:4842).

As used herein “host cell” is a cell which has been transformed or transfected, or is capable of transformation or transfection by an exogenous polynucleotide sequence.

As used herein “isolated” means altered by the hand of man from its natural state, i.e., if it occurs in nature, it has been changed or removed from its original environment, or both. For example, a polynucleotide or a polypeptide naturally present in a living organism is not “isolated,” but the same polynucleotide or polypeptide separated from the coexisting materials of its natural state is “isolated”, as the term is employed herein. As used herein therefore, by way of example only, a transgenic animal or a recombinant cell line constructed with a polynucleotid of the invention makes use of the “isolated” nucleic acid.

### EXAMPLES

Without further elaboration, it is believed that one skilled in the art can, using the preceding description, practice the present invention to its fullest extent. The following detailed examples are merely illustrative and not limitations of the preceding disclosure in any way whatsoever. Those skilled in the art will promptly recognize appropriate variations from the procedures both as to reactants and as to reaction conditions and techniques.

### Example 1

### Cloning and Sequencing of Dog FMO1

Liver tissue obtained from adult male beagle, was cut into manageable pieces, and frozen in liquid nitrogen immediately upon harvest. Total RNA was prepared using Qiagen RNeasy® Mini Kit (Qiagen Inc., Valencia, Calif.). Total RNA concentration was determined by spectrophotometry according to Maniatis (Molecular Cloning, 1989). First strand synthesis was performed using Invitrogen SUPERSCRIPT™ II RNase Reverse Transcriptase (Invitrogen Corporation, Carlsbad, Calif.) with modifications to the manufacturer's suggested protocol. First strand cDNA was synthesized using 400 ng of total RNA and Oligo (dT)15 (Integrated DNA Technologies, Inc., Coralville, Iowa). RNasin (Promega Corporation, Madison, Wis.) was substituted for Invitrogen's RNaseOUT. The reaction was incubated at 42° C. for one hour, heated to 70° C. for 15 minutes, and stored at −20° C. until needed.

Primers were designed from published sequences obtained from Genbank™ for full-length FMOI cDNA from mouse, pig, rabbit, rat, and human. At the five prime end there were only two different variations of sequences downstream to the initiation methionine. Mouse and rat composed one option and pig, rabbit, and human the other. At the three prime end there were as many different sequences upstream to the stop codon as there were sequences. PCR (Polymerase Chain Reaction) reaction was performed using Accuzyme™ DNA Polymerase that also contains three prime to five prime proofreading exonuclease activities (Bioline Ltd, London, U.K.). The only primer combination to generate a full length 1.6 kbp full length cDNA PCR product (the correct size according to other published FMOIs) were the five prime primer from the pig, rabbit, human sequence and the three prime primer based on the pig sequence (forward primer: 5′-AAC ATG GCC AAG CGA GTA; reverse primer: 5′-ACT TAG AGG AAA ATC TGG AAA ATG G). The conditions for the PCR were as follows: 40 cycles including denaturation step at 95° C. for 40s, annealing at 52° C. for 40s, and polymerization at 72° C. for 2min. Followed by an additional 5min at 72° C. The PCR product was visualized by UV light following electrophoresis through a 1% Tris acetate agarose gel containing ethidium bromide. The 1.6 kbp fragment was excised from the gel and the DNA was purified using Bio-Rad's Quantum Prep® Gel Slice Kit (Bio-Rad Laboratories, Hercules, Calif.).

The PCR product was then A-tailed according to a protocol suggested by Promega Corporation and using Biolase™ DNA Polymerase (Bioline Ltd, London, U.K.). The purified A-tailed PCR product was then ligated into a plasmid using Promega's pGEM®-T Easy Vector System. This vector has a three prime terminal thymidine overhang at the insertion site to allow compatibility with PCR products that contain a single deoxyadenosine at the three prime ends of the amplified fragment. This vector conferred ampicillin resistance and contained the lacZ gene to allow for selection of the recombinant clones. The ligation reaction was electroporated into Invitrogen's ElectroMAX™ DHIOB™ cells using a Bio-Rad Gene Pulser™. Electroporated DH10B™ cells were inoculated onto LB agar plates containing ampicillin (100 μg/ml), x-gal (80 μg/ml) and IPTG (0.5mM) and allowed to grow overnight at 37° C. White colonies were harvested, inoculated into liquid culture containing ampicillin (50 μg/ml), and grown overnight at 37° C. in a 300 rpm orbital shaker/incubator. The plasmid DNA was isolated using Qiagen's QIAprep® Spin Miniprep Kit. Five μL of the purified DNA was digested with Not I restriction endonuclease to excise the ligated PCR product from the vector and to confirm successful ligation. The digestion product was visualized by UV light following electrophoresis through a 1% Tris acetate agarose gel containing ethidium bromide. Both strands of the cDNA were twice completely sequenced using a 3700 DNA Sequencer (by Applied Biosystems, Foster City, Calif.) per procedure derived from the manual for the sequencer. The sequences obtained are shown in SEQ ID NO: 1 and SEQ ID NO: 3.

### Example 2

### Subcloning, Expression and Purification of Dog FMO1

The Bac-to-Bac® Baculovirus Expression System (Invitrogen) was used to express the dog FMO1 cDNA in Spodoptera frugiperda (Sf-9) insect cells. Plasmid DNA from the confirmed clone was digested with Not I to extract the full-length 1.6 kbp full-length cDNA. The DNA was ligated into the pFastBac™ vector previously digested with Not I. This placed the dog FMOI cDNA downstream of the polyhedrin promoter. The ligation reaction was electroporated into Invitrogen's ElectroMAX™ DH10B™ cells using a Bio-Rad Gene Pulser™ and plated and allowed to grow overnight at 37° C. on LB agar plates containing ampicillin (100 μg/ml). Colonies were picked and grown in liquid culture containing ampicillin (50 μg/ml) overnight at 37° C. with 300 rpm shaking in an orbital shaker/incubator. The plasmid DNA was isolated using Qiagen's QIAprep® Spin Miniprep Kit. Five μL of the purified DNAs were digested with Spe I restriction endonuclease to select the clones in the correct orientation. The digestion was visualized by UV light following electrophoresis through a 0.8% Tris acetate agarose gel containing ethidium bromide.

The recombinant plasmid DNA was transformed into DH10Bac Escherichia coli cells containing the bacmid DNA. Recombinant bacmid DNA clones were obtained according to Bac-to-Bac® methodology (Invitrogen). PCR was used to verify transposition of the cDNA into the bacmid DNA using one gene specific primer and one bacmid DNA specific primer (M13). The product was visualized by UV light following electrophoresis through a 0.8% Tris acetate agarose gel containing ethidium bromide.

Sf-9 cells were maintained in Grace's Insect Medium with 10% fetal bovine serum containing penicillin G (10 units/ml), streptomycin (10 μg/ml) and amphotericin B (0.25 μg/ml) at 27° C. on an orbital shaker (90 rpm). Cells were transfected with recombinant dog FMOI bacmid DNA with CellFECTIN™ reagent according to the manufacturer's protocol using Grace's Insect Medium without serum during transfection (Invitrogen). Recombinant baculovirus was harvested 72 h post-transfection and amplified in suspension Sf-9 cultures to prepare high titer virus stocks. Titer was determined according to manufacturer's protocol (Invitrogen). Cells were plated on T-150 flasks ( 1×10⁷ cells) and infected with an MOI (multiplicity of infection) of 0.5 in medium supplemented with flavin adenine dinucleotide (FAD) (10 μg/ml). Insect cells were harvested at 72 h postinfection, washed with sucrose buffer containing 280 mM sucrose, 25mM HEPES pH 7.5, 1mM EDTA pH 7.5, 10 μg/ml FAD, and protease inhibitor cocktail (Roche Molecular Biochemicals, Mannheim, Germany, catolog # 1697498). Cells were centrifuged, resuspended in sucrose buffer, and homogenized on ice with a glass-glass homogenizer. The suspension was centrifuged at 1000 g for 10 minutes at 4° C. to remove large cellular debris. The supernatant was then centrifuged at 100,000 g for 80 min at 4° C. to pellet the microsomes. The supernatant was removed and the pellets were thoroughly resuspended in approximately 3 pellet volumes of sucrose buffer supplemented with FAD (10 μg/ml). The suspension was then centrifuged for 1 minute at 1000 g to remove any insoluble material and the supernatant was transferred to a new tube and stored at −80° C. until further use.

The protein concentration of the insect cell microsomes was assayed using bovine serum albumin as a standard (Bio-Rad Laboratories). Ten micrograms of microsomes from the infected insect cells, wild type insect cells, dog liver cells and 2μg of human FMOI Supersomes™ (Gentest Corporation, Woburn, Mass.) as a control, were separated on a 10% tris glycine acrylamide gel by SDS polyacrylamide electrophoresis (SDS-PAGE) (Laemmli, 1970) and transferred to nitrocellulose. The membrane was blocked, and then incubated with human anti-rabbit FMO1 antisera (Gentest Corporation). After washing, the membranes were incubated with horseradish peroxidase-conjugated goat anti-rabbit IgG (Gentest Corporation) as the secondary antibody. FMO1 was visualized in all samples by enhanced chemiluminescence on film.

### Example 3

### Enzyme Activity Assay

MPT S-oxidation has been shown to be a high velocity reaction catalyzed by various FMO forms Sadeque et al., 1992; Rettie et al., 1994; Yeung et al., 2000. An achiral assay method was conducted according to the data sheets provided by Gentest, with some modifications. Based on preliminary determinations of the linearity of metabolite formation with protein concentration and incubation time, incubation conditions were established using 50 μg (tissue microsomes) or 25 μg (Sf-9 cell microsomes) protein, 1 mM NADPH, and 0.1 M tricine in a total volume of 0.25 ml. Samples were incubated for 3 min at 37° C. followed by the addition of substrate (in 1% methanol) to start the reaction. Incubations were terminated after 10 minutes by the addition of 50 μl of acetonitrile, and samples were centrifuged to precipitate protein. The supernatant was analyzed by HPLC, using a 4.6 ×250 mm 5 micron C₁₈ analytical column (Luna™C₁₈(₂), Phenomenex, Torrance Calif.) and a 1 ml/min gradient elution of 40:60/methanol:water to 60:40/methanol/water over 8 minutes, followed by substrate elution with 95% methanol. The MPT sulfoxide product was detected by absorbance at 240 nm and quantitated using the authentic standard. Imipramine N-oxidation was measured by substrate-dependent oxidation of NADPH at 340 nm Wyatt et al., 1998, using 25-50 μg of microsomal protein in a total volume of 750 μl. The expressed polypeptide of SEQ ID NO: 6 of the invention was shown to be active by this assay.

What is claimed is:

1. An isolated, functional dog flavin-containing monooxygenase form 1 polypeptide, wherein the polypeptide is expressed using a baculovirus expression system in insect cells.

2. The polypeptide of claim 1 which comprises the amino acid sequence of SEQ ID NO: 6.

3. The polypeptide of claim 1 which consists essentially of the amino acid sequence of SEQ ID NO: 6.

4. The polypeptide of claim 1 which consists of the amino acid sequence of SEQ ID NO: 6.

5. The polypeptide of claim 1 the amino acid sequence of which comprises a sequence at least 90% homologous with the amino acid sequence of SEQ ID NO: 6.

6. The polypeptide of claim 1 the amino acid sequence of which comprises a sequence at least 95% homologous with the amino acid sequence of SEQ ID NO: 6.

7. The polypeptide of claim 1 the amino acid sequence of which comprises a sequence at least 99% homologous with the amino acid sequence of SEQ ID NO: 6.

8. The polypeptide of claim 1 the amino acid sequence of which differs by up to six amino acids from amino acids of SEQ ID NO: 6.

9. The polypeptide of claim 1 the amino acid sequence of which differs by up to four amino acids from amino acids of SEQ ID NO: 6.

10. The polypeptide of claim 1 the amino acid sequence of which differs by up to two amino acids from amino acids of SEQ ID NO: 6.

11. An isolated polypeptide comprising amino acid sequence of SEQ ID NO: 2 or SEQ ID NO: 4.

12. An isolated polynucleotide comprising a sequence of SEQ ID NO: 1 or SEQ ID NO: 3.

13. The isolated polynucleotide of claim 12 which is DNA.

14. The isolated polynucleotide of claim 12 which is RNA.

15. An isolated polynucleotide comprising a sequence encoding a polypeptide of SEQ ID NO: 2or SEQ ID NO: 4.

16. A vector comprising the polynucleotide of any one of claims 12 to 15, or of SEQ ID NO: 5.

17. A recombinant baculovirus comprising a polynucleotide of any one of claims 12 to 15, or of SEQ ID NO: 5.

18. A recombinant baculovirus of claim 17 comprising the polynucleotide of SEQ ID NO: 5.

19. A host cell containing a recombinant baculovirus of claim 17 or claim 18.

20. The host cell of claim 19 which is an insect cell.

21. The host cell of claim 20 which is a Sf8, Sf9, or Sf21 cell.

22. The host cell of claim 21 which is a Sf9 cell.

23. A host cell of claim 21 containing a recombinant baculovirus of claim 18.

24. A method of metabolizing a sample compound, comprising:
(a) preparing a mixture of the sample compound and the host cell, or cell-free extract thereof, of claim 23,
(b) incubating the mixture and,
( c) optionally analyzing the products obtained thereby.

25. The method of claim 24, further comprising detecting whether the sample compound is detoxified or activated.

26. A method for producing a functional dog FMO1 polypeptide, comprising:
(a) infecting an insect or a cultured insect cell with the recombinant baculovirus of claim 18;
(b) maintaining said insect or said cultured insect cell under conditions wherein said FMO1 polypeptide is expressed; and
(c ) collecting the expressed FMO1 polypeptide.

27. The method of claim 26, wherein said cultured insect cell is an Sf9, Sf21 or a cell derived from Trichoplusia ni.

28. A method for producing a functional dog FMO1, comprising:
(a) generating a recombinant baculovirus comprising the nucleotide sequence of SEQ ID NO. 5;
(b) infecting an insect or a cultured insect cell with the recombinant baculovirus; and
(c) maintaining said insect or said cultured insect cell under conditions wherein said FMO1 is expressed.


SEQUENCE LISTING (6 sequences)

SEQ ID NO: 1 - DNA, 1599 bp, Canis familiaris
atggccaagcgagttgcaattgtgggagctggggtcagtggcctggcctccatcaagtgctgcctggaggaaggactggagcccacatgctttgagaggagtgacgacctcggggggctgtggagattcaccgaacatgttgaaggaggcagagccagcctctacaagtctgtggtttccaacagctgcaaggagatgtcttgttactcagactttcctttcccagaggattatccaaactatgtgccaaattctcaattcctggaatatctcaaaatgtatgcaaaccggttcagccttctgaaatgcattcgattcaagactaaagtctgcaaagtaacaaaatgcccagattttactgtcaccggccaatgggaggtagtcactcagcatgaaggaaagcaagagtctgccatctttgatgctgtcatggtctgcactggtttccttactaacccacatttgccactggactgttttccaggtataaatacatttaaaggccaatactttcatagccgacagtataaacatccagatatatttaaggacaagagagtccttgtgattggaatggggaattcaggcacagacattgctgtagagaccagccgcctggcaaaaaaggtgttcctcagcaccactggaggggcatgggtgatgagccgagtctttgactcaggatacccatgggacgtggtgttcatgactcgatatcagaacatgttcagaaattctctctcaactccaattgtgacgtggttgatggcaaggaagatgaacagctggttcaatcatgcaaattatggcttagtaccagaagacaggacgcagctgagagagcctgtgctaaatgatgagcttccaggctgtatcatcacaggaaaagtgctcataaagccaagcataaaggaggtgaaggaaaattctgttgtatttaacaacaccccaaaggaagagcccattgatatcatcgtctttgccactggatacacctttgctttccccttccttgatgagactgtagtcaaagttgaaaatggccaggcatcgctgtacaagtacatcttccctgtgcatctgccaaaaccaaccctggctgtcattggcctcatcaaacccttaggctccatgatacccacaggagaaacacaagcacgatgggctgttcgagtcctgaaaggtataaataagttaccaccacaaagtgccatgacagaggaagttaacgcaagaaaagaaaacaaacccagtgggtttggcttgtgctactgcaaagctttacaatcagattacatcacatacatagatgaactcctgaccaatatcaatgcaaaacccaacctattctcattgcccctgacggacccacgcctggctttgaccactttctttggcccatgcacaccataccagttccgcttgactggcccagggaaatggaagggagccagaaatgctatcttgactcaatgggatcgaacattcaaagtcaccaaaactcgaattgtacaagaatccccaactccctttgcaagcttgcttaaactcttaagccttctggctttgctaatggccattttccagattttcctctaa

SEQ ID NO: 2 - protein, 532 aa, Canis familiaris
MAKRVAIVGAGVSGLASIKCCLEEGLEPTCFERSDDLGGLWRFTEHVEGGRASLYKSVVSNSCKEMSCYSDFPFPEDYPNYVPNSQFLEYLKMYANRFSLLKCIRFKTKVCKVTKCPDFTVTGQWEVVTQHEGKQESAIFDAVMVCTGFLTNPHLPLDCFPGINTFKGQYFHSRQYKHPDIFKDKRVLVIGMGNSGTDIAVETSRLAKKVFLSTTGGAWVMSRVFDSGYPWDVVFMTRYQNMFRNSLSTPIVTWLMARKMNSWFNHANYGLVPEDRTQLREPVLNDELPGCIITGKVLIKPSIKEVKENSVVFNNTPKEEPIDIIVFATGYTFAFPFLDETVVKVENGQASLYKYIFPVHLPKPTLAVIGLIKPLGSMIPTGETQARWAVRVLKGINKLPPQSAMTEEVNARKENKPSGFGLCYCKALQSDYITYIDELLTNINAKPNLFSLPLTDPRLALTTFFGPCTPYQFRLTGPGKWKGARNAILTQWDRTFKVTKTRIVQESPTPFASLLKLLSLLALLMAIFQIFL

SEQ ID NO: 3 - DNA, 1599 bp, Canis familiaris
atggccaagcgagttgcaattgtgggagctggggtcagtggcctggcctccatcaagtgctgcctggaggaaggactggagcccacatgctttgagaggagtgacgacctcggggggctgtggagattcaccgaacatgttgaagaaggcagagccagcctctacaagtctgtggtttccaacagctgcaaggagatgtcttgttactcagactttcctttcccagaagattatccaaactatgtgccaaattctcaattcctggaatatctcaaaatgtatgcaaaccggttcagccttctgaaatgcattcgattcaagactaaagtctgcaaagtgacaaaatgcccagattttactgtcaccggccaatgggaggtagtcactcagcatgaaggaaagcaagagtctgccatctttgatgctgtcatggtctgcactggtttccttactaacccacatttgccactggactattttccaggtataaatacatttaaaggccaatactttcatagccgacagtataaacatccagatatacttaaggacaagagagtccttgtgattggaatggggaattcaggcacagacattgctgtagagaccagccgcctggcaaaaaaggtgttcctcagcaccactggaggggcatgggtgatgagccgagtctttgactcaggatacccatgggacatggtgttcatgactcgatttcagaacatgttcagaaattctctcccaactccaattgtgacgtggttgatggcaaggaagatgaacagctggttcaatcatgcaaattatggcttagtaccagaagacaggacgcagctgagagagcctgtgctaaatgatgagcttccaggctgtatcatcacaggaaaagtgctcataaagccaagcataaaggaggtgaaggaaaattctgttgtatttaacaacaccccaaaggaagagcccattgatatcatcgtctttgccactggatacacctttgctttccccttccttgatgagactgtagtcaaagttgaaaatggccaggcatcgctgtacaagtacatcttccctgtgcatctgccaaaaccaaccctggctgtcattggcctcatcaaacccttaggctccatgatacccacaggagagacacaagcacgatgggctgttcgagtcctgaaaggtataaataagttaccaccacaaagtgccatgacagaggaagttaacgcaagaaaagaaaacaaacccagtgggtttggctcgtgctactgcaaagctttacaatcagattacatcacatacatagatgaactcctgaccaatatcaatgcaaaacccaacctattctcattgctcctgacggacccacgcctggctttgaccatcttctttggcccatgcacaccataccagttccgcttgactggcccagggaaatggaagggagccagaaatgctatcttgactcaatgggatcgaacattcaaagtcaccaaaactcgaattgtacaagaatccccaactccctttgcaagcttgcttaaactcttaagccttctggctttgctaatggccattttccagattttcctctaa

SEQ ID NO: 4 - protein, 532 aa, Canis familiaris
MAKRVAIVGAGVSGLASIKCCLEEGLEPTCFERSDDLGGLWRFTEHVEEGRASLYKSVVSNSCKEMSCYSDFPFPEDYPNYVPNSQFLEYLKMYANRFSLLKCIRFKTKVCKVTKCPDFTVTGQWEVVTQHEGKQESAIFDAVMVCTGFLTNPHLPLDYFPGINTFKGQYFHSRQYKHPDILKDKRVLVIGMGNSGTDIAVETSRLAKKVFLSTTGGAWVMSRVFDSGYPWDMVFMTRFQNMFRNSLPTPIVTWLMARKMNSWFNHANYGLVPEDRTQLREPVLNDELPGCIITGKVLIKPSIKEVKENSVVFNNTPKEEPIDIIVFATGYTFAFPFLDETVVKVENGQASLYKYIFPVHLPKPTLAVIGLIKPLGSMIPTGETQARWAVRVLKGINKLPPQSAMTEEVNARKENKPSGFGSCYCKALQSDYITYIDELLTNINAKPNLFSLLLTDPRLALTIFFGPCTPYQFRLTGPGKWKGARNAILTQWDRTFKVTKTRIVQESPTPFASLLKLLSLLALLMAIFQIFL

SEQ ID NO: 5 - DNA, 1599 bp, Canis familiaris
atggccaagcgggttgcaattgtgggagctggggtcagtggcctggcctccatcaagtgctgcctggaggaaggactggagcccacatgctttgagaggagtgacgacctcggggggctgtggagattcaccgaacatgttgaagaaggcagagccagcctctacaagtctgtggtttccaacagctgcaaggagatgtcttgttactcagactttcctttcccagaagattatccaaactatgtgccaaattctcaattcctggaatatctcaaaatgtatgcaaaccggttcagccttctgaaatgcattcgattcaagactaaagtctgcaaagtaacaaaatgcccagattttactgtcaccggccaatgggaggtagtcactcagcatgaaggaaagcaagagtctgccatctttgatgctgtcatggtctgcactggtttccttactaacccacatttgccactggactgttttccaggtataaatacatttaaaggccaatactttcatagccgacagtataaacatccagatatatttaaggacaagagagtccttgtgattggaatggggaattcaggcacagacattgctgtagagaccagccgcctggcaaaaaaggtgttcctcagcaccactggaggggcatgggtgatgagccgagtctttgactcaggatacccatgggacatggtgttcatgactcgatttcagaacatgttcagaaattctctcccaactccaattgtgacgtggttgatggcaaggaagatgaacagctggttcaatcatgcaaattatggcttagtaccagaagacaggacgcagctgagagagcctgtgctaaatgatgagcttccaggctgtatcatcacaggaaaagtgctcataaagccaagcataaaggaggtgaaggaaaattctgttgtatttaacaacaccccaaaggaagagcccattgatatcatcgtctttgccactggatacacctttgctttccccttccttgatgagactgtagtcaaagttgaaaatggccaggcatcgctgtacaagtacatcttccctgtgcatctgccaaaaccaaccctggctgtcattggcctcatcaaacccttaggctccatgatacccacaggagaaacacaagcacgatgggctgttcgagtcctgaaaggtataaataagttaccaccacaaagtgccatgacagaggaagttaacgcaagaaaagaaaacaaacccagtgggtttggcttgtgctactgcaaagctttacaatcagattacatcacatacatagatgaactcctgaccaatatcaatgcaaaacccaacctattctcattgctcctgacggacccacgcctggctttgaccatcttctttggcccatgcacaccataccagttccgcttgactggcccagggaaatggaagggagccagaaatgctatcttgactcaatgggatcgaacattcaaagtcaccaaaactcgaattgtacaagaatccccaactccctttgcaagcttgcttaaactcttaagccttctggctttgctaatggccattttcctaattttcctataa

SEQ ID NO: 6 - protein, 532 aa, Canis familiaris
MAKRVAIVGAGVSGLASIKCCLEEGLEPTCFERSDDLGGLWRFTEHVEEGRASLYKSVVSNSCKEMSCYSDFPFPEDYPNYVPNSQFLEYLKMYANRFSLLKCIRFKTKVCKVTKCPDFTVTGQWEVVTQHEGKQESAIFDAVMVCTGFLTNPHLPLDCFPGINTFKGQYFHSRQYKHPDIFKDKRVLVIGMGNSGTDIAVETSRLAKKVFLSTTGGAWVMSRVFDSGYPWDMVFMTRFQNMFRNSLPTPIVTWLMARKMNSWFNHANYGLVPEDRTQLREPVLNDELPGCIITGKVLIKPSIKEVKENSVVFNNTPKEEPIDIIVFATGYTFAFPFLDETVVKVENGQASLYKYIFPVHLPKPTLAVIGLIKPLGSMIPTGETQARWAVRVLKGINKLPPQSAMTEEVNARKENKPSGFGLCYCKALQSDYITYIDELLTNINAKPNLFSLLLTDPRLALTIFFGPCTPYQFRLTGPGKWKGARNAILTQWDRTFKVTKTRIVQESPTPFASLLKLLSLLALLMAIFLIFL